(19) United States
(12) Patent Application Publication
(10) Pub. No.: US 2013/0060100 A1
(43) Pub. Date: Mar. 7, 2013

(54) CONTACTLESS NON-INVASIVE ANALYZER OF BREATHING SOUNDS

(71) Applicants: Arie John Martin Wurm, Ezer (IL); Zeev Heller, Ganei Tikva (IL); Jacob Scheim, Pardess Hanna (IL)

(72) Inventors: Arie John Martin Wurm, Ezer (IL); Zeev Heller, Ganei Tikva (IL); Jacob Scheim, Pardess Hanna (IL)

(73) Assignee: SENSEWISER LTD, GANEI TIKVA (IL)

(21) Appl. No.: 13/697,538

(22) PCT Filed: May 12, 2011
(86) PCT No.: PCT/IL11/00378

§ 371 Date: Nov. 13, 2012

Related U.S. Application Data
(60) Provisional application No. 61/334,202, filed on May 13, 2010.

(51) Int. Cl. A61B 7/04; A61B 5/11; A61B 8/00

(52) U.S. Cl. 600/301

(57) ABSTRACT

A device and method for monitoring and analyzing breathing sounds, the device including at least one microphone adapted and configured for placement adjacent a body to be monitored for contactless recording of breathing sounds, a motion detector using an ultrasound distance sensor to detect the patient body motions, and a processor coupled to the microphones and the motion detector for receiving and processing the breathing sounds and patient motions to detect abnormal breathing events.

### RELATED APPLICATIONS

The present invention claims priority from U.S. Provisional patent application Ser. No. 61/334,202 filed 13 May 2010.

### FIELD OF THE INVENTION

The present invention relates to a method and device for contactless, non-invasive monitoring and detection of body sounds, in general, and breathing sounds, in particular.

### BACKGROUND OF THE INVENTION

Physicians use auscultation as a method for detecting a patient's breathing problems. When used by an experienced physician, this method is very effective for short diagnosis sessions but, due to the direct contact with the patient's body, it becomes inconvenient when a continuous monitoring period is required and is impractical for home use.

Pulmonary diseases, like asthma, involve nocturnal respiratory disruption events manifested by adventitious breath sounds. Detecting these events quickly is important for the immediate treatment of the patient. Providing a continuous events report of days or weeks long sessions is important for assessing the disease state and selecting the appropriate long term treatment. The absence of such reports today compels the physicians to rely on subjective impressions provided by the patients or their relatives.

Most of the current solutions for creating continuous period events reports use stethoscope-like automated sensors. Gavriely in U.S. Pat. No. 6,168,568 describes a phonopneumograph with several sensors placed around the respiratory system of the patient. Those sensors are attached to the patient's body during the monitoring period. These solutions are applicable for short term testing and are primarily for diagnosis by medical staff and cannot be used for continuous and multiple overnight monitoring of asthma patients at home, which is one of the main purposes of the current invention.

Other solutions with contactless sensors, like in U.S. Pat. No. 5,309,921 by Kisner, using infrared sensors to sense CO₂ concentration in the air exhaled by the patient, are tuned for breathing monitoring for sleep apnea and are less appropriate for detecting acoustic breathing events, like coughs and wheezes.

Accordingly, there is a long felt need for a system for providing short and long term breathing events reports, and it would be very desirable for such a system to utilize contactless, non-invasive sensor devices.

### SUMMARY OF THE INVENTION

The current invention proposes a method, system and apparatus for non-invasive monitoring and analysis of breathing sounds without any direct contact with the monitored person. One embodiment of the proposed invention, called herein “Breathing Analyzer” (BA), can monitor, detect and provide an alert of irregular breathing sounds like a cough or wheeze. It can also track, log and display such events detected through multiple sessions for long periods (weeks and months) and it may also support predicting a degradation of the breathing condition.

The BA includes at least one microphone for capturing the monitored person's breathing sounds propagating through the monitored person's environment. The acoustic information received by the microphone/s is processed using signal processing techniques to detect breathing events of interest, like coughs and wheezes. When multiple microphones are used, the BA may also incorporate a sound direction detection process using known methods for direction of arrival analysis, for example, with a microphones array. Sound direction detection may be used to eliminate noise created outside the patient's bed zone.

The BA also includes an ultrasonic distance sensor that is used to detect patient motions, and is capable of measuring delicate motions. The direction, speed and timing information of the patient's motions are correlated with the breathing events detected by analyzing the sounds received through the microphone/s. This correlation supports the detection of the acoustic events and filters out sounds not created by the patient. The motion information can also be utilized for detecting changes in breathing rate and/or breathing interruption events.

The sound information is analyzed further by sound events detector in time and frequency domains to identify irregular breathing events (cough, wheeze and others), as well as breathing characteristics, like breathing rate.

The above mentioned information is processed to create related alerts and events reports. This process, including its embedded calculations and thresholds may be adapted to the monitored environment in order to improve the overall analysis performance and reports quality. The process may refer to, but is not limited to, the following sound characteristics: intensity, pitch, frequency, combination of frequencies, spectrum, duration, source, location, statistical data of the breathing, or any combination of those and others. Once a specific event (cough, wheeze, breathing rate violation or other) has passed corresponding thresholds for alerting, the visual and audio indications for the corresponding event are activated.

Identified abnormal events (cough, wheeze, breathing rate violation, or other), may be recorded in an events log. The events log may be accumulated through multiple sessions and may include events through multiple recording periods up to one month and beyond. The events report, or a subset of the report, and various displays of the events (text, graphic or other) may be delivered to a host computer through a USB or another interface. The report and the displays may be further processed on the host computer and then displayed on the host computer or forwarded to a medical staff for further analysis and assessment of the monitored person's state. The events log, its subsets and various views of them may also be displayed on a local display of the apparatus.

There is provided according to the present invention a device for monitoring and analyzing breathing sounds, the device including at least one microphone placed adjacent, with no direct contact, to a patient's body to be monitored for breathing sounds, a motion detector using an ultrasound distance sensor to detect the patient body motions, and a processor coupled to the microphone/s and the motion detector for receiving and processing the breathing sounds and patient motions to detect abnormal breath events.

There is also provided, according to the invention, a method for monitoring and analyzing breathing sounds, the method including placing at least one microphone adjacent a patient's body to be monitored, recording breathing sounds from the body without contacting the body, detecting motion of the body using an ultrasound distance sensor, and receiving and processing the breathing sounds and patient motions in a processor to detect abnormal breath events.

### BRIEF DESCRIPTION OF THE DRAWINGS

The present invention will be further understood and appreciated from the following detailed description taken in conjunction with the drawings in which:

FIGS. 1a and 1b: Show a typical setup for using the breathing analyzer, according to some embodiments of the invention.

FIG. 2: A block diagram of an analyzer according to one embodiment of the invention, presenting the main elements in the hardware implementation.

FIG. 3: Block diagram of the main data flow in an embodiment of the invention.

FIG. 4: Sound identifier block diagram, presents the operations executed throughout the sound detection process.

FIG. 5: A block diagram describing the analysis process of a single sound event.

FIG. 6: A schematic illustration of a few seconds of a typical sound wave and the marker signal created by the sound identifier during the analysis.

FIGS. 7a and 7b: Two examples of spectrograms created for sample sound events.

FIGS. 8a and 8b: Two sample digitized bi-level images created out of the spectrogram of a cough sound event and a speech sound event

FIG. 9: A block diagram illustrating one embodiment of the frequency domain analysis process.

### DETAILED DESCRIPTION OF THE INVENTION

The present invention relates to a method, system and apparatus for non-invasive and contactless monitoring of respiratory disruption events. This kind of event is common in pulmonary diseases, such as asthma. The method, system and apparatus according to the invention involve detecting and analyzing a subject's respiratory-related sounds, together with corresponding body motion, without any direct contact with the monitored person, and thereby diagnosing the occurrence of respiratory disorder events. The sounds are obtained through the use of one or more microphones, and are coupled with input from an ultrasonic motion detector. The data is analyzed to avoid including noise sources existing in the ambient environment and the output is analyzed further to identify or categorize irregular breathing sounds. All this information is processed using calculations and thresholds to create related alerts and events reports.

The method, system and apparatus may also involve automated or non-automated analysis, real time implementation, alerts and data post processing. The method, system and apparatus may also include supplementary functions and techniques of recording and displaying the processed data.

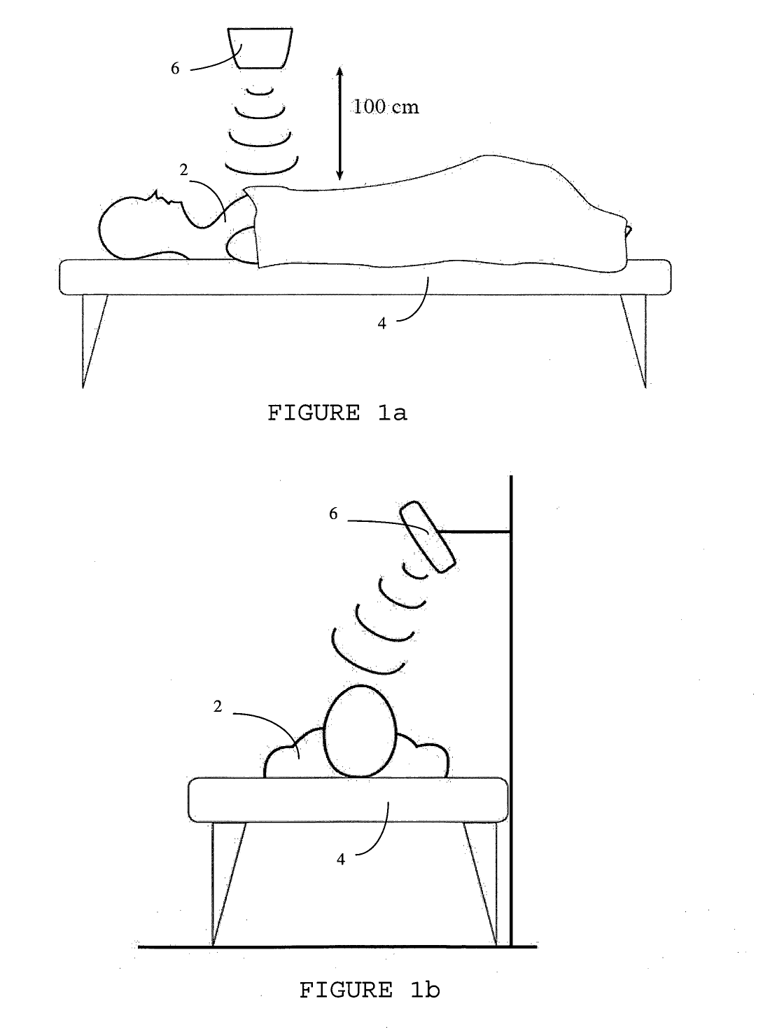

FIGS. 1a and 1b show a typical setup for using the breathing analyzer, according to some embodiments of the invention. A patient 2 lies in a bed 4 while the breathing analyzer device 6 hangs above the bed, preferably pointing towards the patient's torso. It will be appreciated that the device must be mounted far enough from the patient to prevent interference with the patient's movement in the bed, but close enough to collect the necessary data. A distance of up to about 1 meter is preferred.

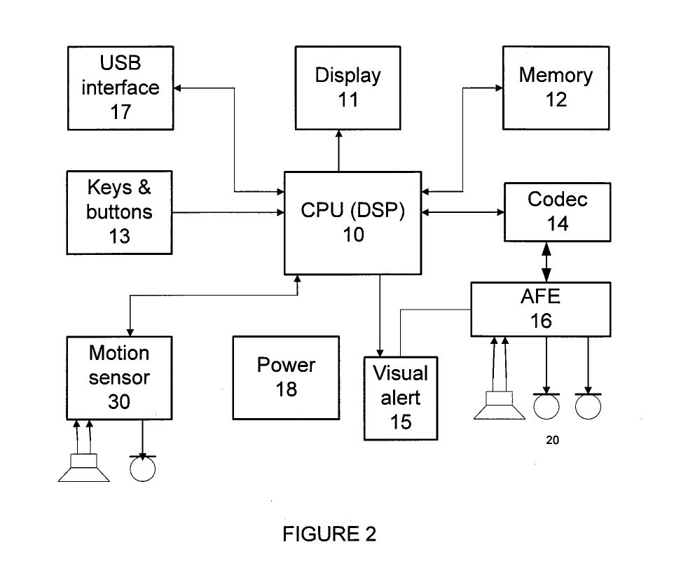

### Detailed 1—Block Diagram—FIG. 2—(Blocks 10,12,14,16,20,22,24, 26,28,30)

FIG. 2 is a block diagram describing the main building blocks of an analyzer according to one embodiment of the invention, presenting the main elements in the hardware implementation. The analyzer includes a CPU 10 that may be of the form of an off-the-shelf digital signal processor (DSP), an application specific processor, a general purpose processor, or any type of computer, and a memory 12 for containing the CPU program, the data, the analysis results and the reports. The system also includes a codec 14, an Analog Front End 16 and microphones 20, for receiving the breathing sounds and a speaker for creating alert sounds. In addition to the above, the system may include also a display 11 (LCD or other), interface keys and buttons 13 and visual alert indicators 15, like LEDs, for operating the device by the user. Alternatively, other user interfaces, like touch display, remote display and control, etc. may be utilized.

The system may include one or more off-the-shelf or custom made interfaces 17 for host computer communications that may be wireless or wired (e.g., USB, Bluetooth, LAN, wireless LAN, etc.). The interface includes the required logic and connector for communicating with a host computer. The system is powered through a power module 18 that can support AC input or DC adapter input or a host driven power connection, like USB power, or batteries. A distance sensor 30 is coupled to CPU 10 for detecting motions. Distance sensor 30 is preferably implemented with an ultrasonic distance meter using an ultrasonic transducer and an ultrasonic microphone. The ultrasonic distance sensor measures the time between ultrasonic sound burst transmission and its echo reception. Changes in the distance are interpreted as motions of the patient and information about motion time, duration, speed and direction is extracted. The ultrasonic distance meter may be connected directly to the CPU, as illustrated, or through sampling and filter units (not shown). The detection zone preferably covers an area the size of a common bed in the direction pointed by the sensor transmitter and receiver. Alternatively, other types of motion sensors, such as an RF sensor or infrared sensors may be utilized, and the motion sensor may be an off-the shelf motion detector or may be specially designed.

Detailed 2—Data Flow Block Diagram

Detailed 2.1—General Description of the Data Flow

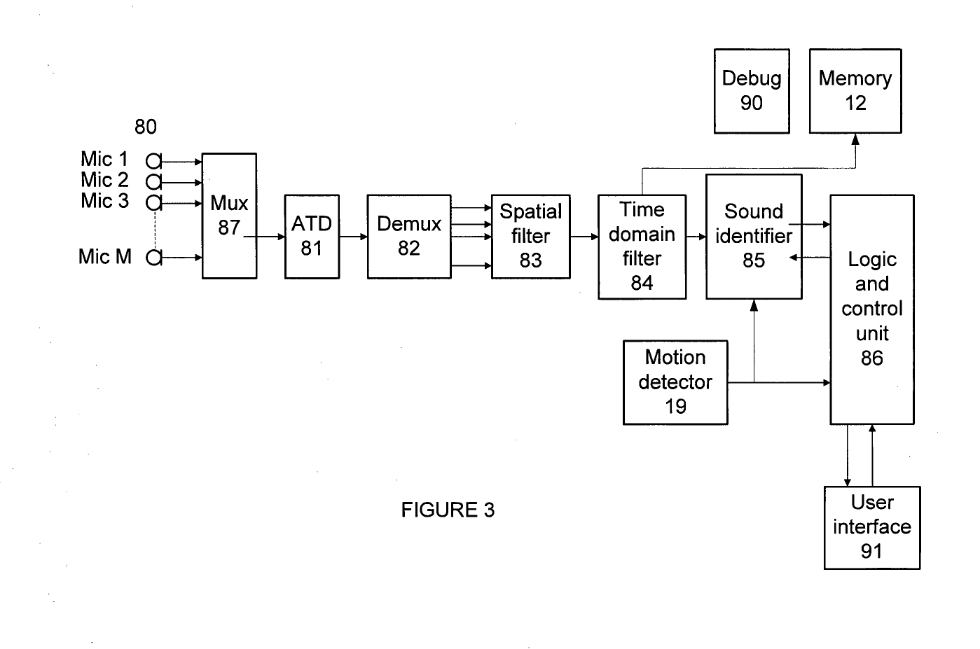

FIG. 3 is a block diagram of the main data flow, according to an embodiment of the invention. Data captured by the microphones and by the motion sensor are processed by the various elements to yield a sound events report. The data flow incorporates two main flows: Acoustic data flow and motions data flow. Acoustic data is received through the open air by one or more microphones—This data is processed to eliminate ambient noise and sounds from sources not related to the patient and to detect specific breathing sounds like cough, wheeze and others. Motion data is received through the open air by a motion detector and reflects the movement of the patient in the bed. Information from both flows is correlated and processed to provide a report of the patient's breathing events, such as cough events, wheeze events and rate, breathing rate and others.

Detailed 2.2—Acoustic Signal Reception—(Block 80)

The acoustic signal is received through one or more microphones 80 located near the monitored person's bed. The number of microphones may be modified as long as it is within the processing capability of the system.

Detailed 2.3 ATD—(Blocks 81,82,87)

An analog to digital signal converter (ATD) 81 converts the signals coming from the microphones into discrete time digital signals. The ATD (block 81) may be a single element serving all the microphones using a multiplexer (Mux) (block 87) and followed by de-multiplexer (DeMux) (block 82), or may be implemented per each single microphone element.

The resulting discrete time digital signal has a meaning of a multi-dimensional signal and is defined a follows:

X(n)=[x ₁(n);x₂(n); . . . ; x_{M}(n)], where n denotes the discrete time phase of sampling, x denotes the microphone output signal, where the subscript (.)_{f} denotes a j^{th} microphone. The samples coming from each microphone are gathered at each time stamp—n, into a vector denoted X(n). In the present setup, M denotes the number of microphones used to measure the sound signal coming from the monitored person.

Detailed 2.4 Spatial Filter—(Block 83)

The digital signals are processed by a central processing unit (CPU). An optional spatial filter (block 83) may be provided for determining the direction from which the sound arrives. Sounds from elsewhere in the environment are filtered out by the spatial filter. The spatial filter may be implemented with known method like microphone array or other custom methods. The spatial filter (block 83) combines a set of samples X. The filter is designed to separate the desired signal from ambient noise and other interfering signals and to amplify the signal coming from a direction of interest out of the entire space (i.e., from the monitored person's direction). The ambient noise may be created by various sources, including instruments, human created sounds and others. The spatial filter (block 83) combines the multi-dimensional signal X into a single-dimension time-domain signal. The resulting time domain signal is the best possible spatial separation between the monitored person's sounds and the other interfering signals and ambient noise.

The spatial filter coefficients may be optimized adaptively to meet a target function. The target function and the optimization process may change according to a higher layer control. However, the rate of change in the filter coefficients is fairly low, since the environment is considered to be quasi-static.

While the use of a spatial filter is optional, its inclusion improves the accuracy of the system.

Detailed 2.5Time Domain Filter—(Block 84)

The next element in the processing chain is a time domain filter (block 84), which further filters the signal in terms of spectral noises and maximizes the signal quality prior to a decision calculation element. One embodiment implements a band pass filter with pass band at 120 Hz:8000 Hz, and −60 db attenuation at the stop bands to eliminate out of band noise (for example, 50 Hz). Other possible implementations of a time domain filter may be used like is a high pass filter. According to another embodiment, no time domain filter is provided at this point in the flow.

Detailed 2.6—Raw Data Storage—(Blocks 84,12)

The filtered signal from the time domain filter is the best achievable quality breathing sound and it is stored in the memory (block 12) as raw data. This data may be used for further analysis, such as debug, medical staff observation and other. The raw data may be transferred from the memory to a larger log space to minimize the required memory size. The log space may also be used as a secondary memory element, for holding the data base of events in a relatively compact size.

Detailed 2.7—Sound Identifier—(Block 85)

The filtered signal then drives a sound identifier (block 85) that analyzes the event both in the time and frequency domains. The detector checks the compliance of the events with timing characteristics of the event, such as minimum and maximum duration. Additionally, the detectors perform frequency analysis of the event by analyzing the regularity of the event spectrogram.

Detailed 2.8—Logic and Control (Block 86,91)

The sound identifier 85 is coupled to a logic and control unit 86. The logic and control unit 86 manages the various processes in the system, activates alerts per predefined policies and controls various system elements. A user interface 91 is provided to permit input of data, instructions, programming, etc. to logic and control unit 86. Some of the system monitoring and control tasks implemented in one embodiment of current invention are:
Initiating spatial filtering adaptive algorithm
System configuration (reset, time, customization and other)
Power and batteries management
Host and device communication (USB)
Reports generation
Recorded data management
User interfade control

Other embodiments of current invention may have additional monitoring and control functions implemented in the logic and control unit or other components of the system.

Detailed 2.7.1—Sound Events Separation and Sound Events Marker Generation—(Blocks 101,102,103,104,105)

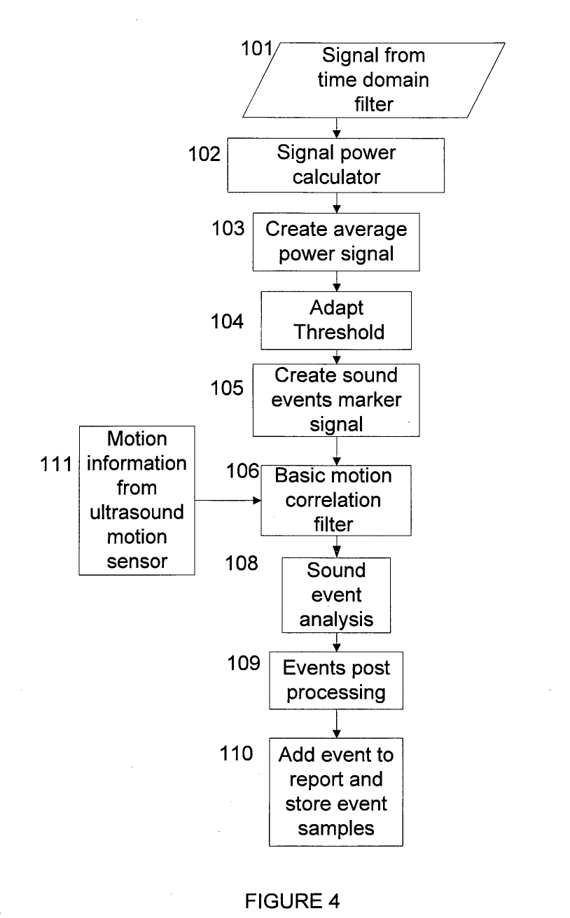

The sound identifier process of block 85 will now be described in more detail with reference to FIG. 4. A signal is received from the time domain filter (block 101). The incoming signal power is calculated in a signal power calculator (block 102) by squaring the samples of the input signal, and an average power signal is calculated (block 103). This signal will be used later to extract individual sounds. The average power signal is created (block 103) by delaying the power signal and averaging through 20 msec. The average power of noticeable sounds is higher than the background noise average power.

A power comparison is used to separate sounds from the background noise. An adapt threshold process (block 104) calculates a threshold to be used for power comparison. The threshold is dynamic and is the square of the standard deviation of the input signal. Other embodiments may elect to use a predefined fixed threshold or a combination of both.

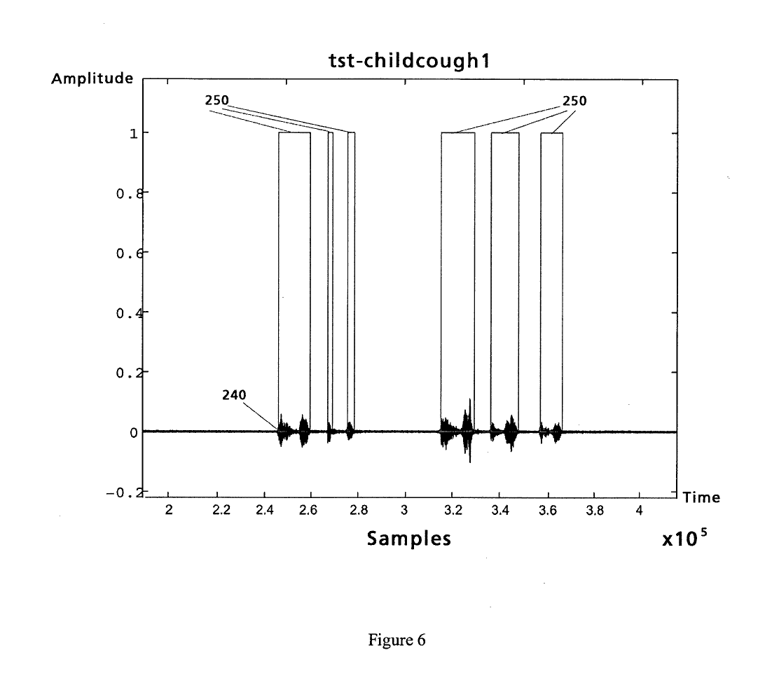

The threshold calculated in (block 104) is used to separate sound events from background noise and create a sound event marker signal (block 105). The marker signal marks where in the input signal window a sound event was detected. A sample image of a sound wave 240 and the sound event marker signal 250 is shown in FIG. 6, a plot of time vs amplitude. A smoothening process through averaging is performed to eliminate ringing of the marker signal at the transition points.

It will be appreciated that other power averaging methods and duration may alternatively be utilized, and longer or shorter signal windows may be used. Furthermore, sound events may be separated from ambient sounds through correlation or other methods.

10.2.7.2 Sound Events and Motion Information Basic Correlation—(Blocks 106,111)

A basic sound and motion correlation is performed (block 106) as shown in FIG. 4, between the sound events and the motions detected within the motion detection zone (block 111) at the same time frame, to determine whether the patient moved at the time of the sound event. If the motion detector detected a movement of the patient within a predefined timing window around the sound event sound samples are transferred forward to the next stage of the main loop. Otherwise, the sound is ignored. This mechanism blocks sounds not generated by the patient but rather by sound sources outside of the detection zone.

The sound movement correlation analysis is based on the physiology involved with the breathing sounds creation. For example, a cough is preceded by a deep inhalation and performed with a quick exhalation, both involving movements of the chest walls that are detected by the motion detector. In a similar way, wheeze sounds are created during inhalation and exhalation of the patient and, thus, involve movement of the chest walls, too, which are detected by the motion detector. Using this mechanism, the system can ignore irrelevant sounds from the surrounding environment that are not related to the breathing motions of the patient.

Detailed 2.7.3—Sound Event Analysis—(Block 108)

Events qualified by the sound event marker and motion information subsequently undergo a sound event analysis (block 108) to identify the specific breathing sound events, such as a cough, wheeze or other. Each individual event marked by a pulse of the sound event marker is analyzed separately.

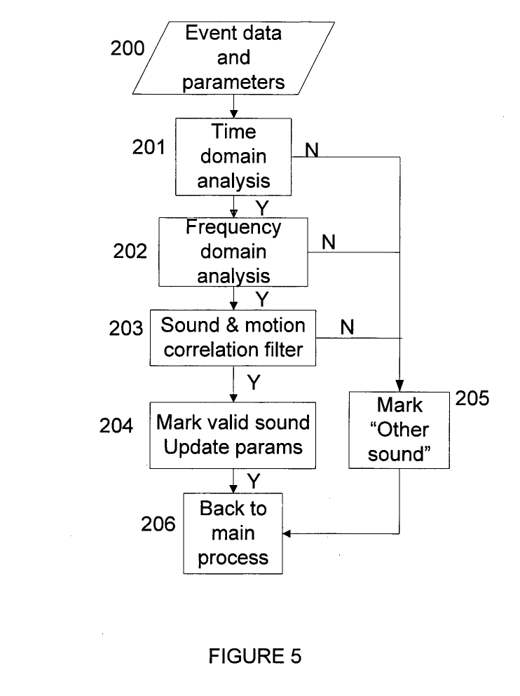

FIG. 5 is a block diagram describing, in detail, the analysis process of a single sound event, according to one embodiment of the invention. Information of the sound event, including signal level and power, event start time and duration, is provided for analysis.

Detailed 2.7.3.1—Sound Event Time Domain Analysis—(Block 200,201)

During this analysis, the event is first checked to comply with several properties in the time domain typical of breathing sound events. The event data and parameters are received (block 200) for time domain analysis (block 201). The properties included in this embodiment are event duration, event rise time from start till average power reached certain percentage of the max average power. In one exemplary embodiment, these properties are cough event length between 0.08-2.5 sec and rise time to 80% of max power. Alternatively, other properties may be utilized, such as events repetition rate, and other values may be selected for the various properties. Events that comply with these checks undergo frequency domain analysis (block 202) for detecting target breathing sound events, including cough, wheeze and other breathing sounds. The processing of events that did not pass the above time domain checks is stopped and they are marked as “other sounds” (block 205).

While it is preferable to implement time domain checks, to reduce the number of false and missed detections, it is possible to implement the method without such time domain checks.

Detailed 2.7.3.2—Sound Event Frequency Domain Analysis—(Blocks 202,300,301,302,303,304,305,306,307,308)

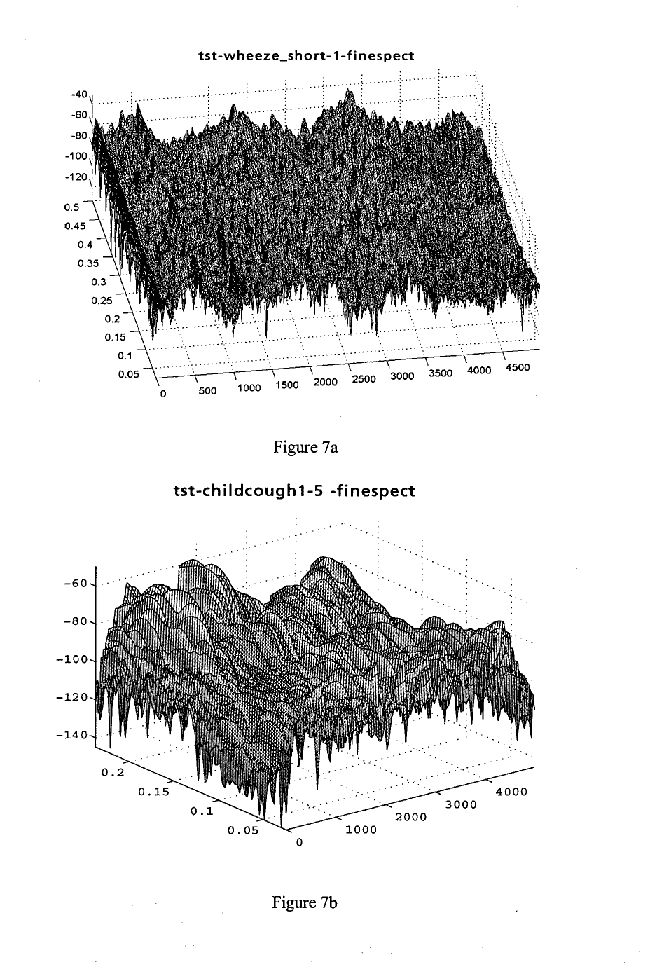

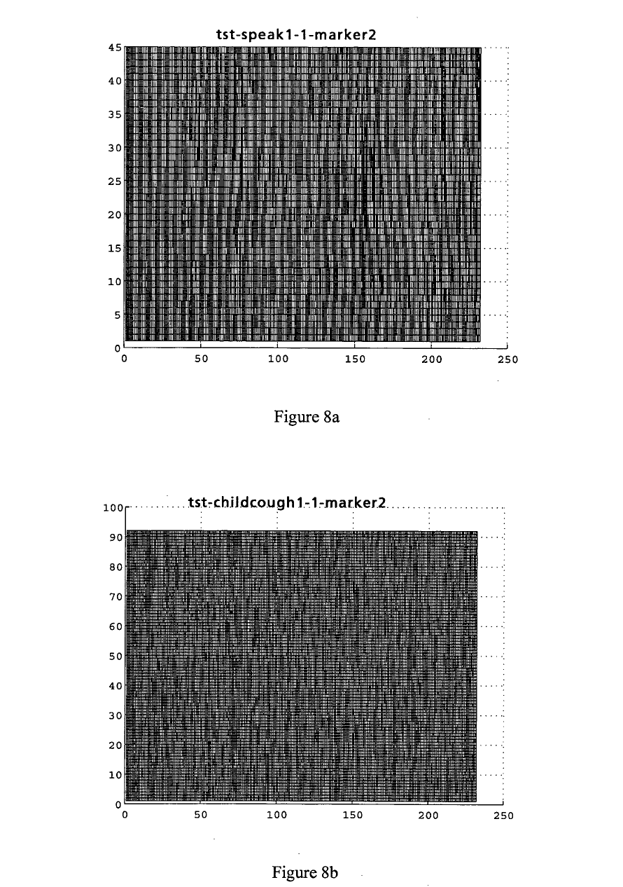

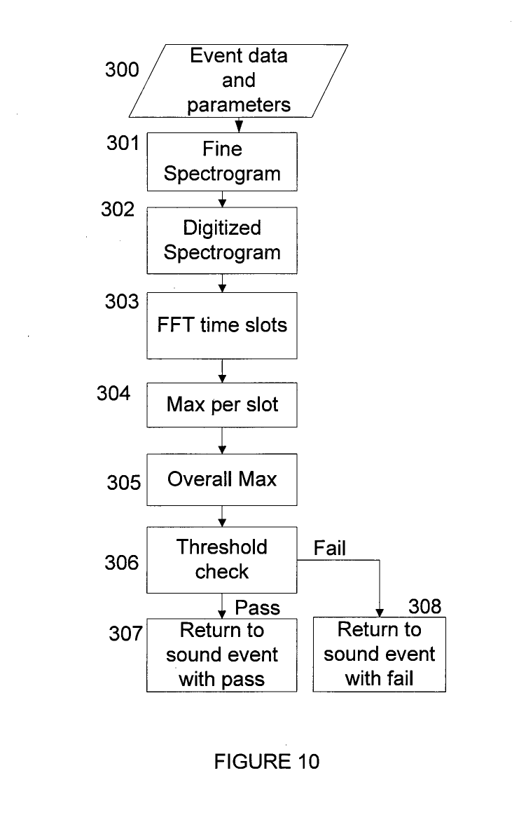

For sound events that pass the time domain analysis, or in the absence of time domain analysis, the data of the sound event now undergoes frequency domain analysis (block 202). The frequency test checks the distribution of the energy or power within a given frequency range during the duration of the sound event. FIG. 9 is a block diagram illustrating one embodiment of the frequency domain analysis process. Information of the sound event being analyzed includes signal level and power, event start time and duration (block 300). A spectrogram of the event is created (block 301) in the frequency range: 0-5000 Hz and throughout the duration of the event. Examples of such a spectrogram are shown in FIGS. 7a and 7b, showing a wheeze and a child's cough, respectively. For a cough detection, the spectrogram is digitized (block 302) using a predefined or dynamic threshold, to provide a bi-level image, as shown in FIGS. 8 and 9, of speech and a cough, respectively. The cough sound energy tends to spread across the frequency vs. time space. Thus, it creates a distributed pattern and the digitized image will have fewer repeating patterns than those encountered in speech, for example, and the image will tend to be evenly distributed, as can be seen in FIG. 8. Other sound types will create their own characteristic images. For example, a speaking voice has a more ordered pattern, so repeating stripes appear in the image. FIGS. 8a and 8b show sample digitized bi-level images created from the spectrogram of a speech and cough sound events. The images are used to determine the identity of the sound event.

The digitized bi-level spectrogram is processed by a Fast Fourier Transform (FFT) (block 303) over each time slot of the digitized bi-level spectrum to identify repeating sequences or patterns. A maximum (max) value of the FFT results for each slot is calculated (block 304), and then an overall max (block 305). The max values relate to the most repeating pattern in each slot and in the overall digitized bi-level spectrogram. The overall max is compared to a pre-defined threshold (block 306), to detect cough sounds.

For wheeze detection, the spectrogram is searched for a pattern of continuous and uniform ridges in several frequencies throughout the event duration. An example of such a pattern is shown in FIG. 7a.

It will be appreciated that various frequency ranges and thresholds for the analysis can be selected. Furthermore, additional or alternative frequency analysis processes can be utilized to detect a cough, such as using a bank of specific band pass filters and also other and additional pattern classification techniques may be used like probabilistic neural networks.

A sound event that does not pass this threshold check is marked as failed (block 308) in FIG. 9 and marked as “other sound” (block 205) in FIG. 5. A sound event that passes the frequency domain check is identified as a valid breathing sound event and returned for further processing (block 307) to FIG. 5.

Detailed 2.7.3.3—Sound and Motion Correlation—(Block 203,204,205,206)

Relating once more to FIG. 5, the sound detection results are now returned to the sound event analysis process. Valid sound events are returned to a sound and motion correlation filter where they are correlated with the motion information from the ultrasound motion detector (block 203). The correlation process determines whether the movements detected are appropriate for the sound event identified, that is, whether the motions characteristics comply with the proposed event type, in terms of speed, timing and direction and/or acceleration.

Events that were correlated successfully with the motions are marked as valid events and their parameters updated accordingly in (block 204). Other sound events are aborted and marked as “other sounds” (block 205). Events data is delivered back (block 206) to the sound identifier process as described in FIG. 4.

Detailed 2.7.4—Events Post Processing—(Block 109)

Confirmed events returning from the sound event analysis go through a post processing process (block 109) as shown in FIG. 4. The events identified earlier in the process are now analyzed to identify special sequences, for example, a main cough followed by sub coughs. The sequences are identified by various parameters, like their duration and the time between the sound events. According to other embodiments of the invention, it is possible to search for and qualify other or additional sequences of interest.

Detailed 2.7.5—Reports Update—(Block 110)

The confirmed and identified events are added to an overall events log maintained in the system or remotely (block 110). The log update accords to a predetermined control policy or policies for recording data.

In case an events sound recording is activated, the recorded event samples are stored in the samples data base. The events samples may be linked with the events log so they can be played later interactively or in a batch by a physician or other users for prognosis or other purposes. The reports may be presented in text or graphically on a local display or on a host or remote system using various User Interfaces or Graphical User Interfaces.

While the invention has been described with respect to a limited number of embodiments, it will be appreciated that many variations, modifications and other applications of the invention may be made. It will further be appreciated that the invention is not limited to what has been described hereinabove merely by way of example. Rather, the invention is limited solely by the claims which follow.

1. A device for monitoring and analyzing breathing sounds, the device comprising:
at least one microphone placed adjacent a body to be monitored for contactless recording of breathing sounds;
a motion detector to detect motions of said body;
a processor coupled to said microphones and said motion detector for receiving and processing said breathing sounds and concurrent detected motions to detect abnormal breathing events.

2. The device according to claim 1, further comprising means for recording said detected breathing events in an events log.

3. The device according to claim 1, wherein said motion detector includes an ultrasonic distance sensor.

4. The device according to claim 1, wherein said processor includes a module for distinguishing said breathing sounds from background sounds.

5. The device according to claim 1, further comprising a spatial filter for determining direction from which sounds are received in said microphones.

6. The device according to claim 1, further comprising a time domain filter for filtering spectral noises from the signal and maximizing the breathing signal quality.

7. The device according to claim 1, further comprising a sound identifier including:
a module for calculating a sound events marker;
a basic motion and breathing sound correlation filter; and
a sound event analyzer.

8. The device according to claim 7, wherein said sound event analyzer includes:
a time domain analyzer;
a frequency domain analyzer; and
a sound and motion correlation filter.

9. The device according to claim 1, wherein said processor is programmed to perform frequency analysis measuring power distribution in a sound event using a bi-level image of the sound event spectrogram and an FFT of the image to detect repeating patterns.

10. A method for monitoring and analyzing breathing sounds, the method comprising:
placing at least one microphone adjacent a body to be monitored;
recording breathing sounds from said body without contacting said body;
detecting motion of said body; and
receiving and processing said breathing sounds correlated with said patient motions in a processor to detect abnormal breathing events of said body.

11. The method according to claim 10, wherein said step of detection motion includes detecting motion of said body using an ultrasound distance sensor.

12. The method according to claim 10, further comprising recording said analyzed breathing sounds in an events log.

13. The method according to claim 10, further comprising distinguishing said breathing sounds from background sounds in said recorded sounds, and analyzing said breathing sounds to detect abnormal breath events including but not limited to coughs wheezes and breathing rate changes.

14. The method according to claim 10, wherein said step of processing includes:
detecting breathing sound events by sound analysis in time and frequency domains; and
correlating said breathing sound events with direction, speed, timing and duration of concurrent body motions detected by said motion detector.

15. The method according to claim 10, further comprising performing frequency analysis measuring power distribution in said breathing sounds using a bi-level image of the breathing sounds spectrogram and an FFT of the image to detect repeating patterns.

16. The method according to claim 15, wherein a cough or wheeze sound is detected by comparing the spectrogram FFT results to predefined thresholds

17. A device for monitoring and analyzing breathing sounds, the device comprising:
at least one microphone, for contactless placement adjacent a body to be monitored, recording breathing sounds from said body;
a motion detector, including an ultrasonic distance sensor, to detect motions of said body;
a processor, implementing time domain analysis and frequency domain analysis of said recorded breathing sounds, coupled to said microphones and said motion detector for receiving and processing said breathing sounds and concurrent detected motions to detect abnormal breath events.

18. The device according to claim 1, wherein said abnormal breathing events are selected from the group including coughs, wheezes and breathing rate changes.

19. The device according to claim 2, wherein said motion detector includes an ultrasonic distance sensor.
